(19) United States
(12) Patent Application Publication
(10) Pub. No.: US 2007/0155668 A1
(43) Pub. Date: Jul. 5, 2007

(54) Peptides modulating the activity of the engrailed transcription factor

(75) Inventors: Alain Prochiantz, Paris (FR); Brigitte Lesaffre, Saint-Denis (FR); Michel Volovitch, Paris (FR); Laure Sonnier, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: Centre National De La Recherche Scient., Paris (FR); Ecole Normale Superieure, Paris Cedex 05 (FR); UNIVERSITE PARIS 13 NORD, Villetaneuse (FR)

(21) Appl. No.: 10/557,239

(22) PCT Filed: May 19, 2004
(86) PCT No.: PCT/FR04/01233

§ 371 Date: Nov. 17, 2005

(30) Foreign Application Priority Data
May 20, 2003 (FR) 03/06023

(51) Int. Cl. A61K 38/10; C07K 7/08; C07H 21/04; C12P 21/06

(52) U.S. Cl. 514/15; 530/328; 435/69.1; 435/320.1; 435/325; 536/23.5

(57) ABSTRACT

The invention relates to peptides which can become fixed to the engrailed transcription factor and which can modulate the activity thereof.

The invention relates to peptides capable of modifying the activity of “Engrailed” transcription factors.

Engrailed proteins are transcription factors of the homeodomain protein class. Mammals possess two Engrailed homologues: Engrailed-1 and Engrailed-2; these two proteins, which have similar activity, will be collectively referred to hereinafter under the general term Engrailed (EN).

In newborns and in adults, EN is expressed in the dopaminergic (DA) neurons of the substantia nigra (which degenerate in Parkinson's disease) and the raphe nuclei and the nuclei of the locus coeruleus, which play an important role in the regulation of mood and the instigating of addictive behavior.

It has been shown (Simon et al. J Neurosci. 21 (9): 3126-34, 2001) that the loss of EN during development is followed quite rapidly by degeneration of dopaminergic neurons, and that one of the transcriptional targets of EN is alpha-synuclein, the genetic linkage of which with certain familial forms of Parkinson's disease has been shown (Polymeropoulos et al., Science. 276, 2045-7, 1997), and which is thought to constitute a negative regulator of dopaminergic transmission (Abeliovich et al., Neuron, 25, 239-52, 2000).

All these observations strongly suggest that Engrailed is involved in neurodegenerative pathologies, and in particular in pathologies that affect dopaminergic neurons, such as Parkinson's disease. Its expression in raphe neurons and locus coeruleus neurons raises the hypothesis of an involvement in other pathologies, such as mood disorders or addiction.

It therefore appears to be particularly desirable to have modulators of Engrailed activity.

The inventors have investigated whether peptides exist that are capable of modulating Engrailed activity by binding to the protein. They have thus identified two peptides that bind to Engrailed, and can specifically modulate its activity in vitro as well as in vivo.

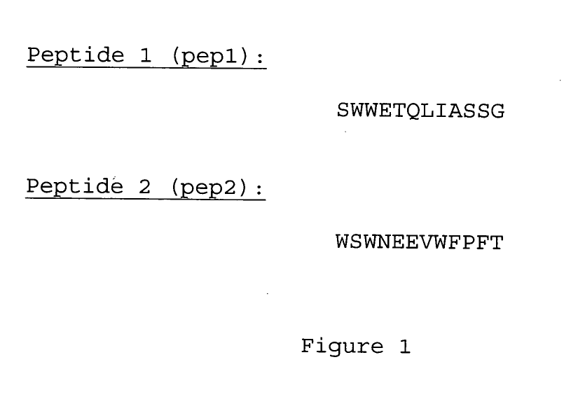

These peptides: SWWETQLIASSG (Peptide 1; SEQ ID No. 1) and WSWNEEVWFPFT (Peptide 2; SEQ ID No. 2) are represented in FIG. 1.

In transformed cells overexpressing Engrailed, peptide 1 has an activating effect, whereas peptide 2 has an inhibitory effect. In a physiological context, both peptides have an activating effect. In addition, the inventors have noted that these peptides confer transcription-activating properties on the homeodomain of Engrailed, which, in itself, does not have these properties.

A subject of the present invention is a peptide capable of binding to the Engrailed transcription factor, and of regulating the activity of said transcription factor.

According to a preferred embodiment of the present invention, said peptide is chosen from the peptides of sequence SWWETQLIASSG and WSWNEEVWFPFT.

A subject of the present invention is also the use of a peptide in accordance with the invention, for regulating the activity of the Engrailed transcription factor in a living cell.

In order to implement the present invention, said peptide may be introduced into the cell in various ways.

Advantageously, it may be combined with a peptide comprising a transducing domain.

The term “transducing domain” denotes a peptide sequence capable of penetrating inside a living cell, independently of the presence of specific transporters or receptors, and capable of importing into said cell molecules or molecular complexes of varied nature (nucleic acids, proteins, peptides/nucleic acids, nucleotide analogues, liposomes), usually referred to under the general term “cargos”.

Transducing peptides of various types are known in themselves. For a review, cf. for example, Lidgren et al., TiPS, 21, 99-102, (2000); Schwarze and Dowdy TiPS, 21, 45-48, (2000); Schwarze et al. Trends Cell. Biol., 10, 290-295, (2000); Prochiantz Current Opinion in Cell Biology, 12, 400-406, (2000); Cell-Penetrating Peptides. Processes and applications. Ed. Ulo Langel. CRC Press (2002).

According to a preferred embodiment of the present invention, a regulatory peptide in accordance with the invention can be combined with a peptide comprising a penetratin-type transducing domain derived from the third helix of a homeodomain; peptides of the penetratin family are described, for example, in the publications by Joliot et al. Proc. Natl. Acad. Sci. USA, 88, 1864-1868, (1991); Derossi et al. J. Biol. Chem., 269, 14, 10444-10450, (1994); Brugidou et al. Biophys. Biochem. Res. Com., 214, 685-693, (1995), and also in U.S. Pat. No. 5 888 762, U.S. Pat. No. 6 080 724, PCT application WO 00/01417 or PCT application WO 00/29427.

A subject of the present invention is also a composition comprising a regulatory peptide in accordance with the invention combined with a peptide comprising a transducing domain, preferably a penetratin-type transducing domain.

According to a preferred embodiment of a composition in accordance with the invention, the regulatory peptide in accordance with the invention is combined with Engrailed, or with a fragment of Engrailed comprising at least its homeodomain.

The combining of these peptides can be carried out by covalent bonding, as described in the documents mentioned above, or else by noncovalent bonding, as described in application FR 03/00093.

The present invention also encompasses any chimeric polypeptide comprising a peptide in accordance with the invention fused to a heterologous peptide. According to a particular embodiment of the present invention, said heterologous peptide comprises at least one transducing domain, preferably a penetratin-type transducing domain. According to a preferred embodiment of a chimeric polypeptide in accordance with the invention, it is a chimeric transcription factor comprising a peptide in accordance with the invention fused to a fragment of Engrailed comprising at least its homeodomain.

A subject of the present invention is also a polynucleotide chosen from:
a polynucleotide encoding a regulatory peptide in accordance with the invention;
a polynucleotide encoding a chimeric polypeptide in accordance with the invention.

The present invention also encompasses a recombinant vector comprising a polynucleotide in accordance with the invention.

The present invention also encompasses a composition comprising a polynucleotide encoding a regulatory peptide in accordance with the invention and a polynucleotide encoding a heterologous peptide, in particular a peptide comprising a transducing domain, preferably a penetratin-type transducing domain. A composition in accordance with the invention may, for example, comprise a recombinant vector comprising a sequence encoding a regulatory peptide in accordance with the invention, and a sequence encoding said heterologous peptide, it being possible for the two sequences to be adjacent or nonadjacent. Alternatively, a composition in accordance with the invention may comprise two different vectors, one carrying a sequence encoding a regulatory peptide in accordance with the invention, and the other carrying a sequence encoding the heterologous peptide.

Vectors comprising a polynucleotide in accordance with the invention may be used, if so desired, for introducing this polynucleotide into and expressing it in the cell in which it is desired to regulate the expression of Engrailed.

With this end, use may be made of the vectors normally used for expressing a gene of interest in animal cells, for example viral vectors. Advantageously, a bacteriophage onto which a transducing peptide is adsorbed, such as those described in application FR 03/00093, can be used as a vector.

The subject of the present invention is also the use of a peptide, or a polynucleotide or of a composition as defined above, for obtaining a medicinal product that can be used in particular in the context of the treatment of nervous system pathologies, for example neurodegenerative pathologies, such as Parkinson's disease.

The present invention will be understood more clearly from the further description which follows, which refers to nonlimiting examples illustrating the obtaining of peptides capable of binding to Engrailed, and of modulating its activity.

### EXAMPLE 1

### Obtaining Peptides Capable of Modulating the Activity of Engrailed

1) System for Testing the Activity of Engrailed

The transcriptional activity of Engrailed is evaluated as described by Montesinos et al. (J. Neurosci., 21, 3350-9, 2001), by measuring the expression of the luciferase reporter gene placed in the plasmid pMAP-luc under the control of a promoter that is a target for Engrailed, the MAP1B (microtubule-associated protein 1B) promoter.

Cells of the neuroepithelial line CHP 100 (Schlesinger et al, Cancer Res. 36, 3904-3100, 1976) are cultured in RPMI 1640 (Life Technologies, Gaithersburg, Md.) supplemented with 16 mM glucose, 15% fetal calf serum, 5 U/ml of penicillin and 5 μg/ml of streptomycin, and cotransfected by electroporation with pMAP-luc and one of the following vectors:

pCL9mEn2 (Mainguy et al., Natural Biotechnol. 18: 746-749, 2000) which contains the sequence encoding the whole chick Engrailed-2 protein, under the control of the CMV promoter;

pCL9mEn2ΔH1 (Joliot et al., Curr Biol. 8: 856-863, 1998), which contains the sequence encoding the chick Engrailed-2 protein from which amino acids 36 to 46 of the homeodomain (DNA-binding sequence) have been deleted, under the control of the CMV promoter;

pCL9mHDEn2C, which contains the sequence encoding the chick Engrailed-2 protein from which amino acids 10 to 185 have been deleted, under the control of the CMV promoter.

The electroporation is carried out with a maximum of h10 μg of plasmid DNA (2 μg of reporter plasmid pMAP-luc+variable amounts of En2 expression vector) per 8×10⁵ cells in 350 μl of medium, at 1050 μF and at 260 mV. 400 μl of medium are then added and the combination is allowed to stand for 10 min. The cells are then placed in culture, after washing, in two dishes (3.5 cm diameter).

The luciferase activity is assayed 24 hours after transfection. The cells are rinsed with PBS. Lysis buffer (20 mM Tris/H₃PO4, pH 7.8, 10 mM MgCl₂, 15% glycerol, 1 mM EDTA, 1% Triton-X 100, 1.25 mM ATP, 10 μg/ml luciferin) is added to the cells. After incubation at 4° C. for 30 min, the luciferase activity is measured by luminometry (Berthold Lumat LB 9501 luminometer).

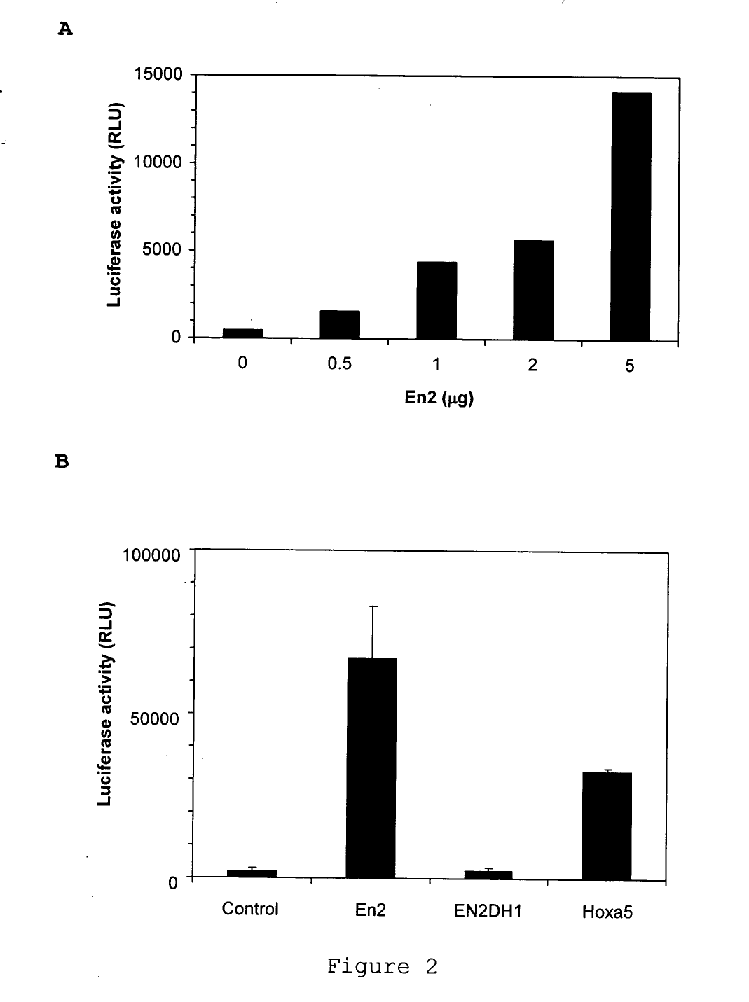

The results are given in FIGS. 2A and 2B.

FIG. 2A shows that the Engrailed-2 (En2) protein is capable of activating the expression of the MAP1B promoter in a dose-dependent manner. FIG. 2B shows that this activation requires binding of the homeodomain to the promoter. The latter is not in fact activated by the EN2AH1 recombinant protein.

The MAP1B promoter is also activated by another homeodomain protein, HOXA5.

2) Selection of Peptides Capable of Binding to Engrailed

Peptides that bind specifically to Engrailed were sought by screening a library of phages (PhD-12, New England Biolabs) expressing random peptides of 12 amino acids at their surface. Several peptides capable of binding to Engrailed were thus obtained.

The two peptides most represented are SWWETQLIASSG (Pep1; SEQ ID No. 1) and WSWNEEVWFPFT (Pep2; SEQ ID No. 2). These peptides were chosen so as to test their effect on the transcriptional activity of Engrailed. The sequences of these peptides are given in FIG. 1.

The sequences encoding Pep1 or Pep2 were placed in the vector pCS2+(Turner and Weintraub, Genes and Development. 8: 1311-1323, 1994), so as to give, respectively, the expression vectors pCS2-pep1 and pCS2-pep2.

3) Test for the Effect of the Peptides Selected on the Activity of Engrailed in CHP100 Cells

The CHP100 cells are cotransfected by electroporation, as defined above, with pMAP-luc (2 μg), pCL9mEn2 or pCL9mHDEn2C (4 μg) and 4 Mg of pCS2-pep1 or pCS2-pep2.

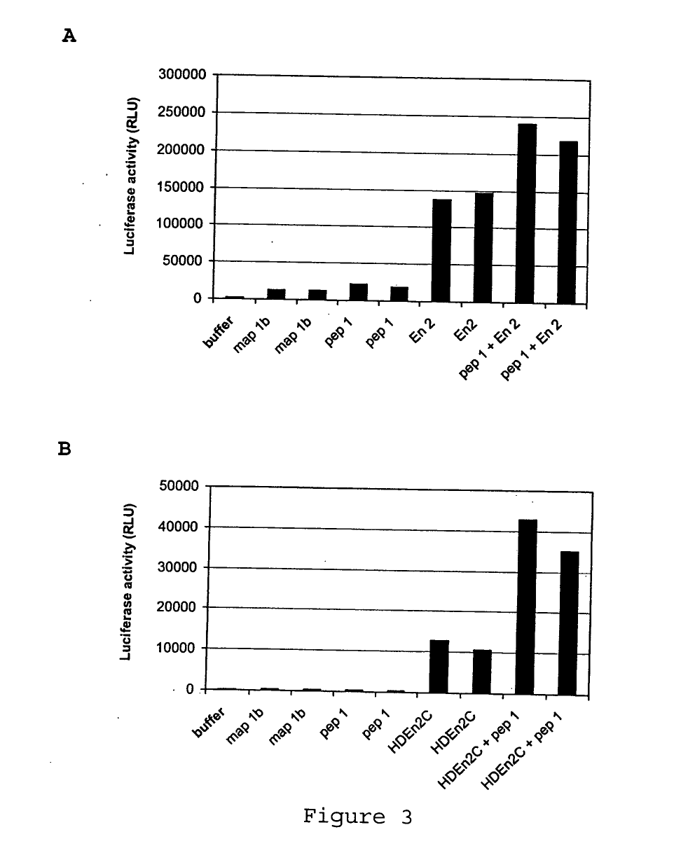

FIG. 3A shows that, when peptide 1 is introduced into the cells at the same time as Engrailed (pep 1+En2), the activation of MAP1B is increased by approximately 50%. The peptide alone has no action on this promoter (pep1). The MAP1B promoter is not active by itself in this cell type (map1b).

FIG. 3B shows that the HDEn2C peptide (consisting mainly of the Engrailed homeodomain) activates only weakly the MAP1B promoter (HDEn2C), and that this activation is greatly increased by peptide 1 (HDEn2C+pep1).

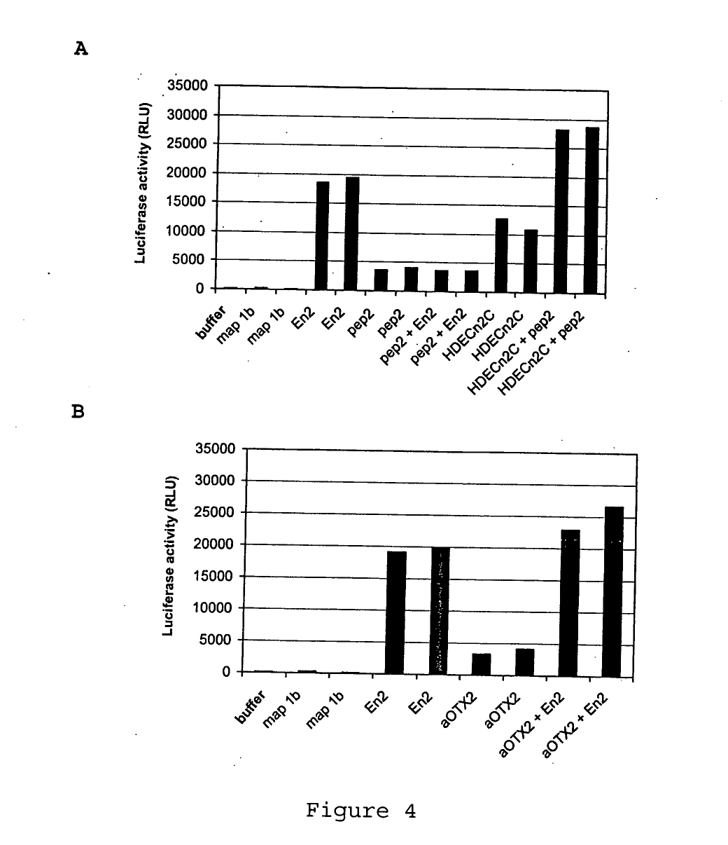

FIG. 4A shows that peptide 2, which has no effect by itself on MAP1B (pep2), is an inhibitor of the activation of MAP1B by Engrailed (pep2+En2), and, conversely, increases the activation of MAP1B by the HDEn2C peptide (HDEn2C+pep2).

FIG. 4B shows that a control peptide (aOTX2) of sequence KVWDIRYTTPHA (SEQ ID No. 3) that binds specifically to the OTX2 homeoprotein does not modify the activation of MAP1B by Engrailed (aOTX2+En2). The effect of the aOTX2 peptide and that of Engrailed are purely additive.

4) Test for the Effect of the Peptide Selected on the Activity of Engrailed in Primary Cultures of Mesencephalic Neurons

In order to verify the functionality of peptides 1 and 2 in a physiological cellular context, the effect of these peptides was tested in primary cultures of mouse embryonic mesencephalic neurons.

Fragments of mouse mesencephalon (embryonic day 13.5) are incubated at 24° C. for 5 min in trypsin-EDTA, then washed in a phosphate buffer, pH 7.5, supplemented with 33 mM glucose (PBS) and 10% fetal calf serum, and subsequently incubated at 37° C. for 10 min in 30 μM/ml of DnaseI (Sigma, Saint Louis, Mo.). The cells are dissociated mechanically, washed three times with PBS, and placed in culture at a density of 200 000 cells/cm², in wells pre-saturated with D,L-poly-ornithine (1.5 μg/ml) and laminin (5 μg/ml). The culture medium (MMS) consists of DMEM/F12 (1/1, Life Technologies, Cergy, France), with 33 mM glucose, 2 mM glutamine, 10 mM HEPES, pH 7.4, 9 mM NaHCO-₃, 5 U/ml of penicillin and 5 μg/ml of streptomycin. Added to MSS are 0.1% of ovalbumin, 25 μg/ml of insulin, 100 μg/ml of transferrin, 20 nM of progesterone, 60 μM of putrescine and 30 nM selenium (M20V).

The day after the cells were placed in culture, the medium, in each well, is replaced with 600 μl of MSS, without penicillin or streptomycin. 100 μl of the transfection mixture comprising pMAP-luc (0.5 μg) and 0.5 μg of pCS2-pep1 or pCS2-pep2, and 1 mg/ml of Lipofectamine 2000 (Invitrogen) in OPTI-MEM medium (qs 100 μl), mixed beforehand for 20 min at 24° C., are added to each well. The cells are then incubated at 37° C. for 2 h 30 min. The medium is then replaced with M20V.

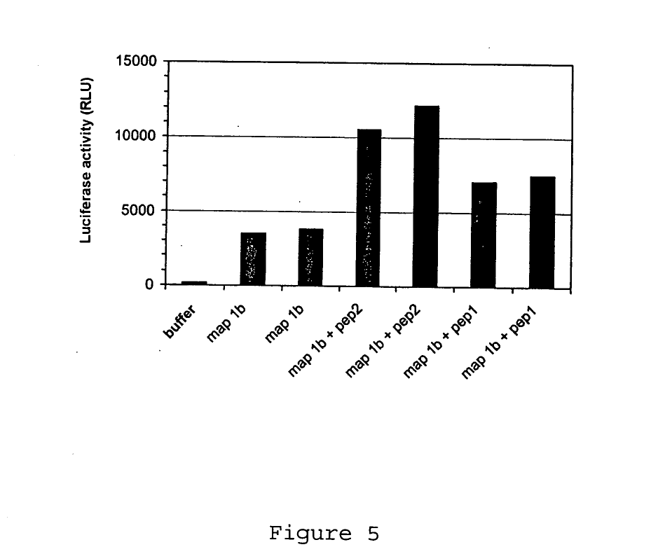

The results are given in FIG. 5.

These results show that the MAP1B promoter is activated in these neurons (map1b), and that this activation is increased by peptide 1 (map1b+pep1) and also by peptide 2 (map1b+pep2), the activating effect of peptide 2 appearing, in this context, to be greater than that of peptide 1.

### EXAMPLE 2

### In Vivo Effect of the Pep-1 and Pep-2 Peptides

The effect of pep1 or pep2 on the transcriptional activity of Engrailed was evaluated by measuring the expression of the β-galactosidase reporter gene placed in the plasmid pMAP-lacZ (described by Montesinos et al., 2001, mentioned above) under the control of the MAP1B promoter, in the neural tube of chick embryos at the HH8-HH10 stage.

The vectors pCS2-pep1 or pCS2-pep2, and also the vector pMAP-lacZ vector (each vector is used at a concentration of 1 μg/μl), were introduced by electroporation into the neural tube of chick embryos at the HH8-HH10 stage in accordance with the protocol of Muramatsu et al. (1997), with a BTX ECM 830 electroporator (4 pulses of 25 V and 50 msec) (Genetronics, San Diego, Calif.). The vectors were injected into the neural tube using a micropipette. After incubation at 37° C. for 24 h, the embryos were fixed and the β-galactosidase activity was revealed, as described by Hogan et al. (Manipulating the mouse embryo. A laboratory manual. Cold Spring Harbor, N.Y.: Cold Spring Harbor laboratory, 1994).

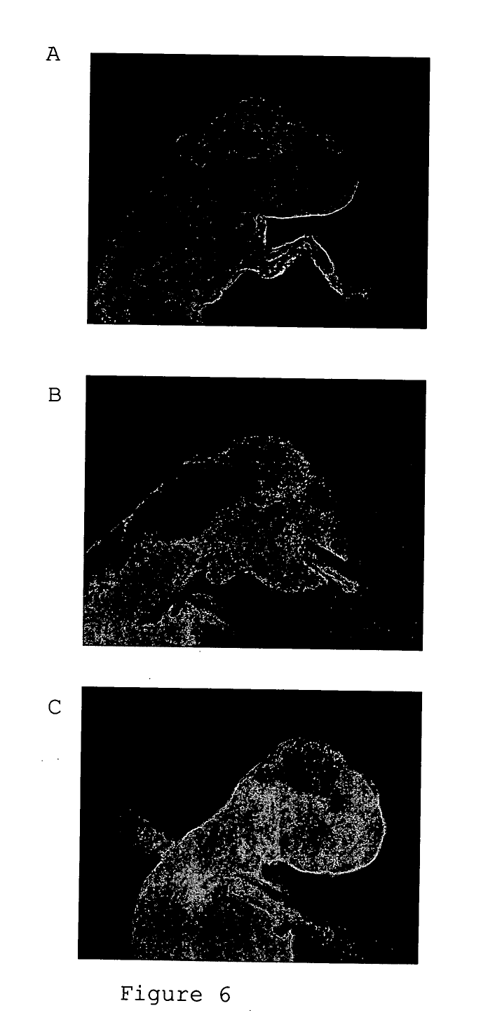

The results are given in FIG. 6 (A-C):

FIG. 6A (pMap-LacZ) shows that the MAP1B promoter is activated in vivo in the mes-metencephalic region corresponding to the normal expression of Engrailed. This expression is confined to the ventral regions, which are the only ones accessible to the plasmids due to the technique of electroporation into the nerve tube.

FIG. 6B (pMap-LacZ +pCS2-Pep1) shows that this activation is increased by peptide 1. The labeling also appears to be more widespread.

FIG. 6C (pMap-LacZ +pCS2-Pep2) shows that peptide 2 also increases the activation of MAP1B.

1. A peptide capable of binding to the Engrailed transcription factor, chosen from:
the peptide of sequence SWWETQLIASSG;
the peptide of sequence WSWNEEVWFPFT.

2. A composition comprising a peptide as claimed in claim 1, combined with a peptide comprising a transducing domain.

3. The composition as claimed in claim 2, characterized in that said transducing peptide is a penetratin.

4. The composition as claimed in claim 3, characterized in that said transducing peptide is the Engrailed protein, or a fragment thereof comprising at least its homeodomain.

5. The composition as claimed in any one of claims 2 to 4, characterized in that it consists of a chimeric polypeptide.

6. A polynucleotide chosen from:
a polynucleotide encoding a peptide as claimed in claim 1;
a polynucleotide encoding a chimeric polypeptide as defined in claim 5.

7. The use of a peptide as claimed in claim 1, of a composition as claimed in any one of claims 2 to 5, or of a polynucleotide as claimed in claim 6, for regulating, in vitro, the activity of the Engrailed transcription factor in a living cell.

8. The use of a peptide as claimed in claim 1, of a composition as claimed in any one of claims 2 to 5, or of a polynucleotide as claimed in claim 6, for obtaining a medicinal product.


SEQUENCE LISTING (3 sequences)

SEQ ID NO: 1 - protein, 12 aa, Artificial Sequence
  Synthetic Peptide
SWWETQLIASSG

SEQ ID NO: 2 - protein, 12 aa, Artificial Sequence
  Synthetic Peptide
WSWNEEVWFPFT

SEQ ID NO: 3 - protein, 12 aa, Artificial Sequence
  Synthetic Peptide
KVWDIRYTTPHA